(19) United States
(12) Patent Application Publication
(10) Pub. No.: US 2010/0261663 A1
(43) Pub. Date: Oct. 14, 2010

(54) Compositions Containing Harpagoside and Paeoniflorin and Methods for Treatment of Conditions Associated with Pain, Inflammation, Arthritis and Symptoms Thereof

(75) Inventor: Alexander R. Shikhman, Escondido, CA (US)

Correspondence Address: CATALYST LAW GROUP, APC, 5694 Mission Center Road, #519, SAN DIEGO, CA, 92108, US

(73) Assignee: Restorative Remedies, San Diego, CA (US)

(21) Appl. No.: 12/756,669

(22) Filed: Apr. 8, 2010

Related U.S. Application Data
(60) Provisional application No. 61/167,668, filed on Apr. 8, 2009.

(51) Int. Cl. A61K 31/7048; A61P 29/00; A61P 19/00; A61P 25/00

(52) U.S. Cl. 514/27

(57) ABSTRACT

The present invention is directed to compositions incorporating purified herbal ingredients for the treatment of conditions such as pain, inflammation, arthritic conditions and other chronic rheumatic diseases, muscle spasms, and headache, as well as methods for their use. The compositions incorporating purified herbal ingredients can incorporate the following combinations of ingredients: (1) harpagoside and paeoniflorin; (2) harpagoside, paeoniflorin, and bioperin; (3) harpagoside and bioperin; and (4) paeoniflorin and bioperin.

This application claims the benefit of priority to U.S. provisional patent application Ser. No. 61/167,668, filed Apr. 8, 2009, entitled “Compositions Containing Harpagoside and Paeoniflorin for Treatment of Pain, Inflammation, Arthritis, and other Conditions,” the disclosure of which is-incorporated herein in its entirety.

### FIELD OF THE INVENTION

This invention is directed to compositions and methods for the treatment pain, inflammation, arthritic conditions and other chronic rheumatic diseases, muscle spasms, and headache. In particular the present invention involves compositions that include harpagoside and paeoniflorin, and optionally include bioperin.

### BACKGROUND OF THE INVENTION

Although many advances have been made in treating conditions such as pain, inflammation, arthritic conditions and other chronic rheumatic diseases, muscle spasms, and headache, these conditions still are responsible for significant deterioration of the quality of life for many patients. In many cases, medications used to treat these conditions are not well tolerated and have significant side effects. For example, many painkillers have been associated with serious gastrointestinal and liver conditions.

Various herbal preparations have been developed for treatment of conditions such as rheumatoid arthritis and other inflammatory conditions. For example, PCT Patent Publication No. WO 2006/082077 by Flavin-Koenig, incorporated herein in its entirety by this reference, discloses a natural remedy-dietary supplement combination product which comprises omega-3 fatty acids, tocopherol (vitamin E) , ascorbic acid (vitamin C), selenium, Harpagophytum procumbens (devil's claw) and Boswellia serrata or carterii (frankincense), with the proviso that it does not comprise a plant or plant extract of the Saxifragaceae family.

Harpagophytum procumbens or rather devil's claw has long been used as a tea by indigenous South African peoples to treat gastrointestinal disorders and rheumatic conditions. A German farmer who had settled in the area exported the plant to Europe where it also became popular among British, European and Canadian herbalists for the supportive treatment of degenerative or rheumatic joint disease, tendonitis and other pains (headache, backache, menstrual pain). It has been also used as an antipyretic, appetite stimulant and bitter tonic, for conditions of the liver, gall bladder and urinary tract, and to treat allergies. An ointment containing devil's claw root is used to treat skin injuries and disorders. Devil's claw has also been shown to be therapeutically effective as treatment for pain.

Paeoniflorin is a glycoside found in root of Ranunculaceae plants. This natural active compound has been commonly used in traditional Chinese medicine. Bioperin or piperine is another naturally occurring compound found in plants of the Piperaceae family. These two compounds have been researched in various studies to uncover possible therapeutic uses for diseases.

Unfortunately, the current therapies for pain inflammation, arthritis, other chronic rheumatic diseases, muscle spasms and headache are either not effective or have deleterious side effects. These treatments are not desirable or suitable for many patients therefore, there is a need for improved compositions and methods to treat these conditions.

### SUMMARY OF THE INVENTION

This invention is based, at least in part, on the discovery that harpagoside and paeonliflorin have therapeutic properties. These therapeutic properties are useful for the prevention and/or treatment of diseases associated with pain. The present invention relates to treating conditions in mammals such as pain, inflammation, arthritic conditions and other chronic rheumatic diseases, muscle spasms, and headache by administering a pharmaceutical composition containing combinations of two or more of harpagoside, paeoniflorin, and optionally bioperin.

Therefore, one embodiment of the present invention is a pharmaceutical composition comprising:
(1) a therapeutically effective quantity of harpagoside; and
(2) a therapeutically effective quantity of paeoniflorin, in a unit dose.

Typically, for this embodiment and other embodiments that are pharmaceutical compositions, the pharmaceutical composition further comprises a pharmaceutically acceptable carrier, diluent, or excipient.

In general, the pharmaceutical composition typically comprises from about 1 mg to about 500 mg of harpagoside and from about 1 mg to about 1000 mg of paeoniflorin as a unit dose.

The pharmaceutical composition can be formulated for oral, transcutaneous, parenteral, or intraarticular routes of administration. Typically, the pharmaceutical composition is formulated for the treatment of a disease or condition selected from the group consisting of pain, inflammation, arthritic conditions and other chronic rheumatic diseases, muscle spasms, and headache.

Another embodiment of the present invention, as described above, includes bioperin in a therapeutically effective quantity in the unit dose. In general, this embodiment comprises:
(1) a therapeutically effective quantity of harpagoside;
(2) a therapeutically effective quantity of paeoniflorin; and
(3) a therapeutically effective quantity of bioperin, in a unit dose.

Typically, the therapeutically effective quantity of bioperin is typically from about 0.1 mg to about 20 mg per unit dose.

Yet another embodiment of the present invention is a pharmaceutical composition incorporating harpagoside and bioperin. In general, this pharmaceutical composition comprises:
(1) a therapeutically effective quantity of harpagoside; and
(2) a therapeutically effective quantity of bioperin, in a unit dose.

Still another embodiment of the present invention is a pharmaceutical composition incorporating paeoniflorin and bioperin. In general, this pharmaceutical composition comprises:
(1) a therapeutically effective quantity of paeoniflorin; and
(2) a therapeutically effective quantity of bioperin, in a unit dose.

Still another embodiment of the present invention is a purified preparation of harpagoside that comprises at least 45% harpagoside.

Still another embodiment of the present invention is a method for treating a patient who has a condition selected from the group consisting of pain, inflammation, arthritic conditions and other chronic rheumatic diseases, muscle spasms, and headache comprising the step of administering a therapeutically effective dose of a pharmaceutical composition according to the present invention, as described above, to the patient to treat the patient.

It is contemplated that any method or composition described herein can be implemented with respect to any other method or composition described herein.

Unless otherwise defined, all technical and scientific terms used herein have the same meaning as commonly understood by one of ordinary skill in the art to which this invention belongs. Methods and materials are described herein for use of the present invention; other suitable methods and materials known in the art can also be used. The materials and methods, and examples are illustrative only and not intended to be limiting. All publications, patent applications, patents and other references mentioned herein, are incorporated by reference in their entirety. In case of conflict, the present specification, including definitions will control.

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows a representative time course of plasma levels of harpagoside after oral administration of 162 mg harpagoside (1800 mg Harpagophytum extract).

FIG. 2 shows the maximal concentration (Cₘₐₓ) of harpagoside in human whole blood samples after different doses of Harpagophytum extract.

FIG. 3 shows the area under the curve (AUC) from 0 to 24 hours. Blood samples from volunteers in study 1 through 3 were taken after oral intake of Harpagophytum extract at the time points indicated in Table 1. AUC(0-t) (circles) and AUC(0-∞) (inverted triangles).

FIG. 4 shows the biotransformations of paeoniflorin (PF) into paeonimetabolin-I (PM-I) and thio-paeonimetabolin-I (PT-PM-I) carried out by intestinal bacteria in rat feces.

### DETAILED DESCRIPTION OF THE INVENTION

### Terms

In accordance with the present invention and as used herein, the following terms are defined with the following meanings, unless explicitly stated otherwise. These explanations are intended to be exemplary only. They are not intended to limit the terms as they are described or referred to throughout the specification. Rather, these explanations are meant to include any additional aspects and/or examples of the terms as described and claimed herein.

The following terms are used herein:

The term “active ingredient” refers to a therapeutically effective amount of drug or formulation thereof Preferably, active ingredients of the present invention are harpagoside, paeoniflorin and bioperin.

The term “therapeutically effective amount” refers to the amount of an active ingredient necessary to induce one or more of the desired pharmacological effects of the current invention. The amount can vary greatly according to the effectiveness of a particular active substance; the age, weight, and response of the individual; as well as the nature and severity of the individual's symptoms. Accordingly, there is no upper or lower critical limitation with respect to the amount of the active substance. A therapeutically effective amount to be employed in the present invention can readily be determined by those skilled in the art.

The term “arthritis” refers to any particular disease characterized by joint inflammation, although the etiology of the inflammation may differ in various conditions. Relatively common arthritic diseases include rheumatoid arthritis, juvenile arthritis, ankylosing spondylitis, psoriatic arthritis and osteoarthritis.

The term “in combination with” as used herein means that the described agents can be administered to a subject together in a mixture, concurrently or as a single agents or sequentially as single agents in any order.

The term “preventing” refers to reducing the likelihood that the recipient will incur or develop any of the pathological conditions described herein.

The term “subject” means any mammal including humans.

The term “treating” refers to mediating a disease or condition and preventing, or mitigating, its further progression or ameliorate the symptoms associated with the disease or condition.

### Materials and Methods

### Mosby Pain Scale

A pain scale rating is a helpful tool in determining how much pain a person is feeling. The Mosby Pain scale was used to determine the level of pain each subject was experiencing at a given point in time. Patients were told to provide a pain scale for each week during the 4 week study. The Mosby Pain Scale instructions are as follows. Explain to the person that each face is for a person who feels happy because he has no pain (hurt) or sad because he has some or a lot of pain. Face 0 is very happy because he doesn't hurt at all. Face 1 hurts just a little hit. Face 2 hurts a little more. Face 3 hurts even more. Face 4 hurts a whole lot. Face 5 hurts as much as you can image, although you don't have to be crying to feel this bad. Ask the person to choose the face that best describes how he is feeling.

Methods and materials are described herein. However, methods and materials similar or equivalent to those described herein can be also used to obtain variations of the present invention. The materials, methods, and examples are illustrative only and not intended to be limiting.

It has been discovered that the herbal ingredients harpagoside, paeoniflorin and bioperin have unique therapeutic properties. The present invention is directed to compositions incorporating these purified herbal ingredients for the treatment of conditions such as pain, inflammation, arthritic conditions and other chronic rheumatic diseases, muscle spasms, and headache, as well as methods for their use. The compositions incorporating purified herbal ingredients can incorporate the following combinations of ingredients: (1) harpagoside and paeoniflorin; (2) harpagoside, paeoniflorin, and bioperin; (3) harpagoside and bioperin; and (4) paeoniflorin and bioperin.

Harpagoside is a biologically active substance present in the plant known as devil's claw (Harpagophytum procumbens), which is a plant native to the red sand areas in the Transvaal of South Africa and Namibia. It has spread throughout the Kalahari and Savannah desert regions. The plant's secondary tubers contain a number of biologically active substances. The most commonly used and well-characterized one is an iridoid glycoside (a glycoside is any molecule in which a sugar group is bonded through its anomeric carbon to another group via an O-glycosidic bond or an S-glycosidic bond; the sugar group is then known as the glycone and the non-sugar group as the aglycone or genin part of the glycoside) harpagoside.

The structural formula of harpagoside is Formula (I), below.

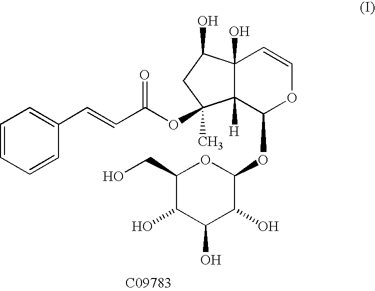

The biological effects of harpagoside in vitro that are pertinent to arthritis and allied conditions include inhibition of 5-lipoxygenase synthesis, inhibition of human leukocyte elastase, inhibition of thromboxane B₂ synthesis, inhibition of NF-κB activation and corresponding inhibition of iNOS and COX-2 expression, inhibition of inflammatory responses in mesangial cells, inhibition of IL-1β-induced matrix metalloproteinase production in human articular chondrocytes, and inhibition of TNF-α synthesis in LPS-stimulated primary human monocytes. Additionally, harpagoside has anti-malarial activity, antioxidant activity, and suppresses contractile responses of small intestinal explants to acetylcholine and other agents.

In vivo, harpagoside exhibits a number of biological effects, including anti-inflammatory and analgesic activity in animals with adjuvant-induced arthritis, inhibition of skin inflammatory responses, mild hypotensive activity, and antiarrhythmic activity.

The pharmacology of harpagoside and Harpagophytum extracts has been studied. The biological activity of devil's claw extract and harpagoside in particular is significantly reduced by the acidic pH of gastric secretions. Harpagoside was shown to be transformed into the pyridine monoterpene alkaloid aucubinine B by human intestinal microflora.

The pharmacokinetic parameters of harpagoside were observed in the 3 studies after oral administration of different amounts of Harpagophytum extract. The Cₘₐₓ values in human whole blood were reached within 1.3 to 2.5 hours and were calculated as 15.4, 16.4, 8.2, 32.2, 27.8, and 50.1 ng/mL corresponding to the doses 43.8, 100, 108, 150, 162, and 200 mg harpagoside, respectively (Table 1).

TABLE 1
Pharmacokinetic parameters after single oral administration of different doses of
Harpagophytum extracts

| | Dose (mg harpagoside/mg extract) | | | | | |
|---|---|---|---|---|---|---|
| | 43.8/600 | 100/400 | 108/1200 | 150/600 | 162/1800 | 200/800 |
| | (study 1) | (study 1) | (study 3) | (study 2) | (study 3) | (study 1) |
| No. of human male volunteers | 1 | 1 | 3 | 6 | 3 | 1 |
| Cₘₐₓ (ng/mL) | 15.4 | 16.4 | 8.2 | 32.2 | 27.8 | 50.1 |
| tₘₐₓ (h) | 2 | 2.5 | 1.3 | 1.3 | 1.8 | 2.5 |
| AUC(0-t) (ng · h/mL) | ND | 65.9 | 17.2 | 157.5 | 160.5 | 225.7 |
| AUC(0-∞) (ng · h/mL) | ND | 82 | ND | 226 | 187.9 | 355.8 |
| Terminal t_{1/2} (h) | ND | 3.7 | ND | 5.6 | 4.4 | 6.4 |
| CL (L/min) | ND | 20.3 | ND | 12.2 | 14.4 | 9.4 |

Cₘₐₓ, Peak concentration;
tₘₐₓ, time to reach Cₘₐₓ;
AUC, area under the plasma concentration-time curve;
t_{1/2}, half-life:
ND, not determined.

The t_{1/2} was short and ranged between 3.7 and 6.4 hours. The clearance is about 15 L/min. FIG. 1 shows a representative time course of plasma levels of harpagoside after oral administration of 162 mg harpagoside (1800 mg Harpagophytum extract from study 3).

FIG. 1 shows the time course of the plasma levels of harpagoside after oral administration of Harpagophytum extract. Blood samples from 3 volunteers were taken at the indicated time points after oral intake of 1800 mg Harpagophytum extract, which represents a harpagoside content of 162 mg. The data represent the mean ±SD (n=3).

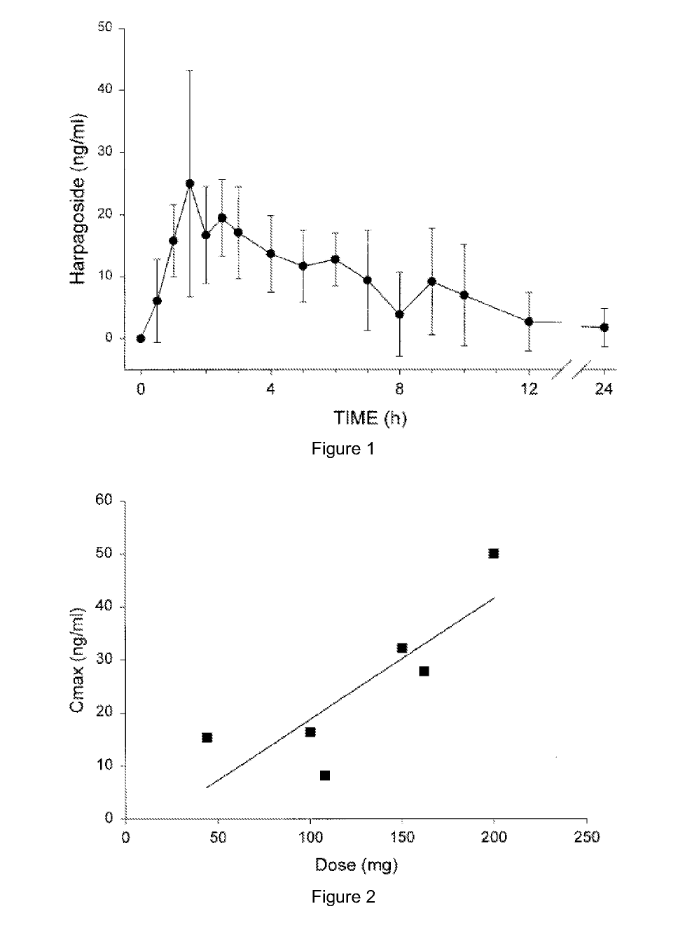

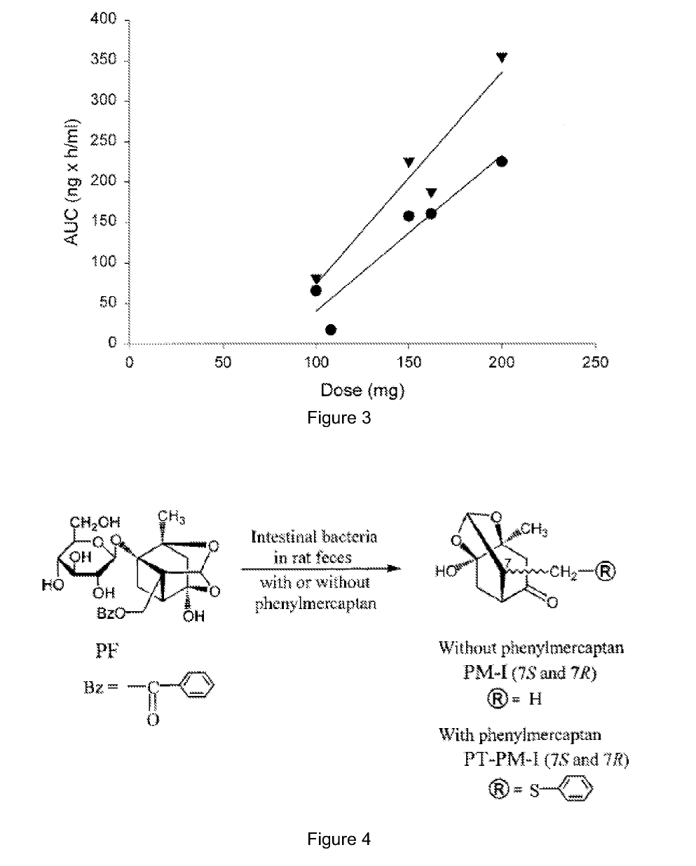

The mean maximum level of harpagoside was detected after 1.8 hours. There was a linear relationship between close and Cₘₐₓ (FIG. 2) or dose and AUC(0-t)/AUC(0-∞) (FIG. 3), which was significant for the AUC values (r2 >0.91; P=0.95) but not for Cₘₐₓ (r²=0.69). After administration of 600 mg Harpagophytum extract in study 3, which reflects 54 mg harpagoside, no harpagoside was detected in whole blood of the volunteers. As shown in FIGS. 2 and 3, harpagoside blood levels were measurable only with a dose higher than 54 mg, which may be the result of a considerable first-pass effect or a low oral absorption.

FIG. 2 shows the maximal concentration (Cₘₐₓ) of harpagoside in human whole blood samples after different doses of Harpagophytum extract.

FIG. 3 shows the area under the curve (AUC) from 0 to 24 hours. Blood samples from volunteers in study 1 through 3 were taken after oral intake of Harpagophytum extract at the time points indicated in Table 1. AUC(0-t) (circles) and AUC(0-∞) (inverted triangles).

With regard to the toxicity, carcinogenicity, and mutagenic activity of harpagoside, harpagoside is not listed in EPA's Toxic Substances Control Act (TSCA) Inventory (NLM, 2000a).

No reports of occupational exposure to harpagoside during its production or processing were found in the available literature. No listing was found for harpagoside in the National Occupational Exposure Survey (NOES), which was conducted by the National Institute for Occupational Safety and Health (NIOSH) between 1981 and 1983.

No standards or guidelines have been set by NIOSH or OSHA for occupational exposure to or workplace allowable levels of harpagoside. Harpagoside was not on the American Conference of Governmental Industrial Hygienists (ACGIH) list of compounds for which recommendations for a threshold limit value (TLV) or biological exposure index (BEI) are made.

No epidemiological studies or case reports investigating the association of exposure to harpagoside and cancer risk in humans were identified in the available literature. No 2-year carcinogenicity studies of harpagoside were identified in the available literature.

The LD₅₀ in mice is greater than 13.5 grams per kg of body weight. The data on chronic harpagoside activity in animals are absent. The data on long-term use of devil's claw extracts in human (over 12 months) indicate very low toxicity. No mutagenic activity of harpagoside has been described in the literature.

The examples below demonstrate devil's claw efficacy when treating joint pain and inflammation.

Warnock M. et al. analyzed the effect of the devil's claw extract on joint pain and function in a heterogeneous group of patients with various rheumatic conditions. 259 patients were treated for 8 weeks in an open labeled clinical trial. There were statistically significant (p<0.0001) improvements in patient assessment of global pain, stiffness and function as compared to the baseline parameters. There were also statistically significant reductions in mean pain scores for hand, wrist, elbow, shoulder, hip, knee and back pain. Quality of life measurements (SF-12) were significantly increased from baseline and 60% patients either reduced or stopped concomitant pain medication.

Chrubasik S. et al recruited 114 patients (56 with chronic nonspecific low back pain, 37 with osteoarthritic knee and 21 with osteoarthritic hip pain) into a surveillance of the effects of taking devil's claw extract at a dose providing 60 mg harpagoside per day for up to 54 weeks. Their symptoms and well-being were monitored at 4-6 week intervals by disease-specific and generic outcome measures, and the patients also kept a diary of their pain and requirement for rescue medication. The principal analyses were on the basis of Intention to Treat (ITT) with Last Value Carried Forward (LOCF). A Multivariate Analysis of Variance (MANOVA) indicated an appreciable overall improvement during the surveillance, similar in the Back, Knee and Hip groups. In separate ANOVAs, most of the individual outcome scores decreased significantly over time. Multiple regression analyses indicated that changes from baseline were independent of patients' characteristics. Additional analgesic requirements (which were very modest) declined during the year of surveillance. “Response during treatment,” assessed according to criteria adapted from joint proposals of the Outcome Measures in Rheumatoid Arthritis Clinical Trials group and the Osteoarthritis Research Society International group, was achieved in 75% of patients, and was reflected in the percentages who rated the treatment as “good” or “very good.” Adverse events were few and none were serious.

Chrubasik S. et al analyzed the efficacy of devil's claw extract taking for one year in patients who were previously recruited into a 6-week double-blind pilot study comparing between 44 devil's claw patients and 44 rofecoxib patients being treated for acute exacerbations of chronic low back pain. Therefore, 38 “ex-Doloteffin” (ex-D) and 35 “ex-rofecoxib” (ex-R) received the extract containing 60 mg harpagoside per day for up to 54 weeks. Pain, additional analgesics, mobility, general health and adverse events were assessed from diary records and at 6-week interval visits. 53 patients remained in the follow-up at 24 weeks and 43 at 54 weeks. There was never any significant difference between ex-D and ex-R patients in the number of patients remaining in follow-up, diary pain scores, additional analgesics, Arhus Index and health assessment questionnaire scores (HAQ). Individual fluctuations notwithstanding, the follow-up showed a slight overall improvement on the improvements in Arhus and HAQ scores achieved in the pilot study (MANOVA p=0.016). Of the 21761 patient-days, the respective percentages with no, mild, moderate, severe and excruciating pain were 28%, 39%, 22%, 8.5% and 1.5%, respectively. Few patients requested additional treatments for their pain. Three patients suffered from minor adverse drug reactions.

Chrubasik S. et al enrolled 250 patients suffering from nonspecific low back pain (Back group: n=104) or osteoarthritic pain in the knee (Knee group: n=85) or hip (Hip group: n=61) and treated them for 8-weeks with devil's claw extract providing 60 mg of harpagoside per day. The measures of effect on pain and disability included the percentage changes from baseline of established instruments (Arhus low hack pain index*, WOMAC index*, German version of the HAQ**) and unvalidated measures (total pain index*, three score index*, the patient's global assessment** of the effectiveness of treatment). Patients also received a diary for the daily recording of their pain and any additional treatments for it. The three groups differed in age, weight and characteristics of initial pain. 227 patients completed the study. Multivariate analysis confirmed that several dimensions of effect were recorded by the several outcome measures but, in all groups, both the generic and disease-specific outcome measures improved by week 4 and further by 8. In multivariable analysis, the improvement tended to be more when the initial pain and disability score was more: older patients tended to improve less than younger, the hip group tended to improve convincingly more than the back group, whereas the improvement in the knee group was less readily differentiated from that in the back group. The subgroup of Back patients who required NSAIDs during the 8 weeks used significantly more per patient than patients in the other two groups, but that requirement also declined more with time. About 10% of the patients suffered from minor adverse events that could possibly have been attributable to the devil's claw. Between 50% and 70% of the patients benefitted from the devil's claw with few adverse effects.

Chantre P. et al performed a double-blind, randomized, multicentre clinical study focused on the efficacy and tolerance of a herbal medicine product Harpadol (6 capsules/day, each containing 435 mg of powdered cryoground powder Harpagophytum procumbens) in comparison with diacerhein 100 mg/clay in the treatment, for 4 months, of 122 patients suffering from osteoarthritis of the knee and hip. Assessments of pain and functional disability were made on a 10 cm horizontal visual analogue scale; severity of osteoarthritis was evaluated by Lequesne's index. Spontaneous pain showed a significant improvement during the course of the study and there was no difference in the efficacy of the two treatments. Similarly, there was a progressive and significant reduction in the Lequesne functional index and no statistical difference was found between Harpadol and diacerhein. At completion of the study, patients taking Harpadol were using significantly less NSAIDs and antalgic drugs. The frequency of adverse events was significantly lower in the Harpadol group. The most frequent event reported was diarrhea, occurring in 8.1% and 26.7% of Harpadol and diacerhein patients respectively. The global tolerance assessment by patients at the end of treatment favored Harpadol. The results of this study demonstrate that Harpadol is comparable in efficacy and superior in safety to diacerhein.

Leblan D. et al evaluated the efficacy and safety of devil's claw in the treatment of hip and knee osteoarthritis comparatively with the slow-acting drug for osteoarthritis, diacerhein. A multicenter, randomized, double-blind, parallel-group study was conducted in 122 patients. Treatment duration was four months and the primary evaluation criterion was the pain score on a visual analog scale. Devil's claw extract 2,610 mg per day was compared with diacerhein 100 mg per day. After four months, considerable improvements in osteoarthritis symptoms were seen in both groups, with no significant differences for pain, functional disability, or the Lequesne score. However, use of analgesic (acetaminophen-caffeine) and nonsteroidal anti-inflammatory (diclofenac) medications was significantly reduced in the Harpagophytum group, which also had a significantly lower rate of adverse events.

Chrubasik S. et al compared two daily doses of devil's extract (600 and 1200, respectively, containing 50 and 100 mg of the marker harpagoside) with placebo over 4 weeks in a randomized, double-blind study in 197 patients with chronic susceptibility to back pain and current exacerbations that were producing pain worse than 5 on a 0-10 visual analogue scale. The principal outcome measure, based on pilot studies, was the number of patients who were pain free without the permitted rescue medication (tramadol) for 5 days out of the last week. The treatment and placebo groups were well matched in physical characteristics, in the severity of pain, duration, nature and accompaniments of their pain, the Arhus low back pain index and in laboratory indices of organ system function. A total of 183 patients completed the study. The numbers of pain-free patients were three, six and 10 in the placebo group (P), the Harpagophytum 600 group (H600) and the Harpagophytum 1200 group (H1200) respectively (P=0.027, one-tailed Cochrane-Armitage test). The majority of responders were patients who had suffered less than 42 days of pain, and subgroup analyses suggested that the effect was confined to patients with more severe and radiating pain accompanied by neurological deficit. However, subsidiary analyses, concentrating on the current pain component of the Arhus index, painted a slightly different picture, with the benefits seeming, if anything, to be greatest in the H600 group and in patients without more severe pain, radiation or neurological deficit. Patients with more. pain tended to use more tramadol, but even severe and unbearable pain would not guarantee that tramadol would be used at all, and certainly not to the maximum permitted dose. There was no evidence for Harpagophytum-related side-effects, except possibly for mild and infrequent gastrointestinal symptoms.

Wegener, T. and Lüpke, N. P. conducted an open-label multicenter study analyzing the efficacy of devil's claw extract (2400 mg extract daily, corresponding to 50 mg harpagoside) in 75 patients with osteoarthritis of the hip or knee for 12 weeks. To standardize the assessment of treatment effects, the Western Ontario and McMaster Universities (WOMAC) osteoarthritis index (10 point scale) as well as the 10 cm VAS pain scale were used. The results of the study revealed a strong reduction of pain and the symptoms of osteoarthritis. There was a relevant improvement of each WOMAC subscale as well as of the total WOMAC index: 23.8% for the pain subscale, 22.2% for the stiffness subscale and 23.1% for the physical function subscale. The WOMAC total score was reduced by 22.9%. VAS pain scores were decreased by 25.8% for actual pain, 25.2% for average pain, 22.6% for worst pain and 24.5% for the total pain score. The physicians reported a continuous improvement in typical clinical findings such as 45.5% for pain on palpation, 35% for limitation of mobility and 25.4% for joint crepitus. Only two cases of possible adverse drug reactions were reported (dyspeptic complaints and a sensation of fullness). Although this was an open clinical study, the results suggest that this Devil's claw extract has a clinically beneficial effect in the treatment of arthrosis of the hip or knee.

Göbel, H. et al conducted a randomized, double-blind, placebo controlled study focused on devil's claw effects on sensory, motor and vascular mechanisms of muscle pain. In addition to clinical efficacy and tolerability, possible action mechanisms were analyzed by means of experimental algesimetric methods. The study was performed on patients with slight to moderate muscular tension or slight muscular pain of the back, shoulder and neck. A total of 31 patients in the devil's claw group and 32 in the placebo group were recruited into the study. The treated group received 2×1 film tablets per day, i.e. 2×480 mg/day, of Harpagophytum extract at 8.00 a.m. and 8.00 p.m. over 4 weeks. Data recording at 14-day intervals was made using a visual analogue scale, pressure algometer test, recording of antinociceptive muscular reflexes, muscle stiffness test, EMG surface activity, muscular ischemia test, clinical global score and subjective patient and physician ratings. After four weeks of treatment there was found to be a clear clinical efficacy of the devil's claw on the clinical global score and in the patient and physician ratings. Highly significant effects were found in the visual analogue scale, the pressure algometer test, the muscle stiffness test and the muscular ischaemia test. No difference from placebo was found in the recording of antinociceptive muscular reflexes or in the EMG surface activity. Tolerability was good; no serious adverse effects occurred. It was concluded that treatment with devil's claw extract has a significant influence on sensory and vascular muscular response and reduces muscle stiffness. No central nervous effects were discovered.

Laudahn, D. and Walper, A., performed an open, multicenter study to evaluate the clinical effectiveness and tolerance of the devil's claw extract in patients suffering from non-radicular back pain over a period of at least 6 months. A total of 130 patients were treated twice a day with tablets containing 480 mg of the extract. The treatment lasted for 8 weeks. The effectiveness was judged according to the Multidimensional Pain Scale (MPS), Arhus back pain index and to parameters evaluating the mobility of the lumbar spine (finger-floor distance, Schober's sign). Data from 117 patients were evaluated for efficacy. The results showed a significant improvement of pain symptoms and mobility of the affected sections of the patient's spine in the course of treatment. No serious side effects were observed. In view of the excellent compliance and tolerability the investigated extract appears to be an effective plant alternative for the treatment of chronic back pain.

Other described clinical effects of devil's claw include improvement in constipation, diarrhea, appetite and flatulence.

Contraindications for the use of devil's claw are few. They include gastric and duodenal ulcers, biliary tree obstruction/chronic calculous cholecystitis, pregnancy, or lactation.

Adverse reactions associated with the use of devil's claw are typically mild. They include allergic reactions, gastrointestinal upset, headache, tinnitus, anorexia, and loss of taste.

Potential drug interactions include possible interactions with coumadin and anti-arrhythmic drugs.

Regarding potential clinical applications in rheumatology for devil's claw, devil's claw extract is a dietary supplement. Since 1994, dietary supplements have been regulated under the Dietary Supplement Health and Education Act (DSHEA). The DSHEA requires no proof of safety for dietary supplements on the market prior to Oct. 15, 1994. Labeling requirements for dietary supplements allow warnings and dosage recommendations as well as substantiated “structure or function” claims. All claims must prominently note that they have not been evaluated by the FDA, and they must bear the statement “This product is not intended to diagnose, treat, cure, or prevent any disease” (FDA. 1995).

Based on the known biological effects and published human data, devil's claw extract can be used to control pain in patients with various joint diseases including but not limited osteoarthritis, rheumatoid arthritis, ankylosing spondylitis/spondyloarthropathies and low back pain.

The following references are directed to the use of extracts of devil's claw for the treatment of various conditions:

Baghdikian, B., et al., Formation of nitrogen-containing metabolites from the main iridoids of Harpagophytum procumbens and H. zeyheri by human intestinal bacteria. Planta Med. 1999 March; 65 (2):164-6. Warnock, M., Effectiveness and safety of Devil's Claw tablets in patients with general rheumatic disorders. Phytother Res. 2007 December; 21(12):1228-33. Chrubasik., S., et al., Patient-perceived benefit during one year of treatment with Doloteffin. Phytomedicine. 2007 June; 14(6):371-6. Chrubasik, S., et al., A 1-year follow-up after a pilot study with Doloteffin for low back pain. Phytomedicine. 2005 January; 12(1-2):1-9. Chrubasik, S., et al., Comparison of outcome measures during treatment with the proprietary Harpagophytum extract doloteffin in patients with pain in the lower hack, knee or hip. Phytomedicine. 2002 April; 9(3):181-94. Chantre, P., et al., Efficacy and tolerance of Harpagophytum procumbens versus diacerhein in treatment of osteoarthritis. Phytomedicine. 2000 June;7(3):177-83. Leblan D, et al., Harpagophytum procumbens in the treatment of knee and hip osteoarthritis. Four-month results of a prospective, multicenter, double-blind trial versus diacerhein. Joint Bone Spine. 2000; 67(5):462-7. Wegener T and Lüpke N P., Treatment of patients with arthrosis of hip or knee with an aqueous extract of devil's claw (Harpagophytum procumbens DC.). Phytother Res. 2003 December; 17(10):1165-72. Gabel H., et al., Effects of Harpagophytum procumbens LI 174 (devil's claw) on sensory, motor and vascular muscle reagibility in the treatment of unspecific back pain] Schmerz. 2001 February; 15(1):10-8. Laudahn D. and Walper A., Efficacy and tolerance of Harpagophytum extract LI 174 in patients with chronic non-radicular back pain. Phytother Res. 2001 November; 15(7):621-4. Zimmerman W., Pflanzliche Bitterstoffe in der Gastroenterologie. Z. Allgemeinmed 1976; 54:1178-84.

Paeoniflorin is a glycoside from the peony (Paeonia lactiflora) root. Its chemical name is 5β-[(Benzoyloxy)methyl]tetrahydro-5-hydroxy-2-Methyl-2,5-methano-1H-3,4-dioxacyclobuta[cd]pentalen-1α(2H)-yl-β-D-glucopyranoside.

The structural formula of paeoniflorin is Formula (II), below.

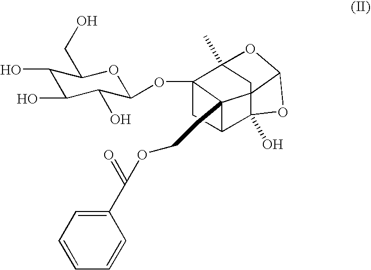

The biological effects of paeoniflorin in vitro that are pertinent to arthritis and allied conditions include anti-inflammatory activity via induction of T-lymphocyte apoptosis (1); immunoregulatory activity at the level of the local intestinal immune system (2); and anti-fibrotic activity via inhibition of TGFβ₁ (3) (numbers in parentheses in this and following paragraphs refer to numbered references at the end of the discussion of paeoniflorin).

Other biological effects of paeoniflorin include anti-oxidant and anti-mutagenic activity (4), blockade of neuronal L-type Cat channels (5), and stimulation of glucose transport (6).

The in vivo biological effects of paeoniflorin include anti-arthritic activity (7), reduction of TNFα and 1L-6 expression (8), muscle relaxing activity (9), mild glucocorticoid and mineralcorticoid activities (10), vasodilation (11), anti-thrombotic effect (12), photoprotection (13), hepatoprotection (3,8), gastroprotection via inhibition of hydrochloric acid secretion (14), glucose-lowering activity (15), cholesterol-lowering activity (16), neuroprotection (17), analgesic activity and antinociceptive activity via binding to adenosine Al receptor (18), anti-Parkinsonian activity (19), anti-epileptic activity (20), enhancement of cognitive activity (21), and restoration of age-related and stroke-related decline in memory and learning ability (22, 23).

The pharmacology of paeoniflorin and peony extracts has been studied. However, the data on pharmacology of paeoniflorin in humans is limited. In animal studies, the absorption and excretion of paeoniflorin after intravenous and oral administration was studied in rats to evaluate the significance of paeoniflorin in the pharmacological action of peony root. The plasma concentration of paeoniflorin after intravenous administration at the doses of 0.5, 2.0 and 5.0 mg kg⁻¹ rapidly decreased, simulated by a biexponential curve, with mean terminal half-lives of 11.0, 9.9 and 12.6 min, respectively. The V_{dss} values were 0.332, 0.384 and 0.423 L kg⁻¹ and the CLₜₒₜ values were 26.1, 31.2 and 30.3 mL min⁻¹ kg⁻¹ at each dose. When given orally at the same doses, the absolute bioavailability values (F) determined by the AUC were 0.032, 0.033 and 0.038, respectively. The cumulative urinary and fecal excretions of paeoniflorin at the dose of 5 mg kg ⁻¹ after intravenous administration were 50.5 and 0.22% of the dose within 72 h, and 1.0 and 0.08% of the dose after oral administration within 48 h, respectively. Cumulative biliary excretion after intravenous or oral administration at a dose of 0.5 mg kg⁻¹ was 6.9 and 1.3% of the dose within 24 h, respectively. The total CLR and CLB value after intravenous dosing was less than the value. These findings suggest that paeoniflorin is metabolized in other organs as well as in the liver. It was concluded that paeoniflorin absorbed is excreted mainly in urine, it has a low bioavailability and the metabolites may be involved in the pharmacological action of peony root (24).

Orally administered paeoniflorin is transformed into bioactive metabolites by intestinal bacteria before they are absorbed into the blood. It has been described that paeonimetabolin-1 (PM-1) and thio-paeonimetabolin-1 (PT-PM-1) are biotransformed from paeoniflorin (PF) by intestinal bacteria in rat feces (25). These biotransformations are shown in FIG. 4.

The extraction ratios of paeoniflorin in gut wall (EG), liver (EH) and lung (EL) were assessed by comparing AUCs after various routes of its administration to estimate the first-pass effects and the metabolism by intestinal flora. Pulmonary extraction ratio of paeoniflorin was assessed by comparing AUCs calculated from venous and arterial plasma concentrations after its intravenous administration (0.5 mg kg⁻¹). The mean pulmonary extraction ratio was estimated to be 0.06. The hepatic extraction ratio (EH was assessed by comparing AUCs after intraportal and intravenous administrations (0.5 and 5 mg kg⁻¹). The plasma concentration profiles of paeoniflorin after intraportal administration were very close to those after intravenous administration, suggesting a negligible hepatic extraction ratio of paeoniflorin. The AUC value after intraperitoneal administration (0.5 mg kg⁻¹) was greater than that after intraportal or intravenous administration. This finding suggests that paeoniflorin is not metabolized in the gut wall. The transference of paeoniflorin from the serosal side to the mucosal side was evaluated by the in-vitro everted sac method. The low intestinal permeability (1.9.4% at 60 min) was demonstrated by the comparison with phenobarbital (63.1% at 60 min). It was concluded that paeoniflorin is not metabolize by gut wall, liver and lung, its poor absorption from the intestine results in extremely low bioavailability and the unabsorbed fraction of paeoniflorin is degraded by the intestinal flora (26).

Quantification of paeoniflorin in mice plasma following oral administration of peony root extract was achieved by using a simple and rapid high-performance liquid chromatographic method. The calibration curve for paeoniflorin was linear (r²=0.998) over the concentration range 10-200 ng/ml. The coefficients of variation of intra- and inter-day assays were 15,04, 7.31, 6.14, 6.55, 6.63% and 12.71, 6.07, 3.61, 5.51, 4.52% at concentrations of 10, 60, 100, 160, 200 ng/ml, respectively. The recoveries of paeoniflorin from mice plasma were found robe 74.49, 76.83, 80.38 and 80.56% for concentrations of 30, 80, 120 and 160 ng/ml, respectively. The plasma concentration-time curves were fitted with mean terminal half-lives (t_{(1/2)}) of 94.16 min (27).

Paeoniflorin has been investigated for activity in the adjuvant arthritis model in rats (Y. Q. Zheng et al., “Effects and Mechanisms of Paeoniflorin, a Bioactive Glycoside from Paeony Root, on Adjuvant Arthritis in Rats,” Inflammation Res. 56: 182-188 (2007), incorporated herein by this reference.)

With regard to the toxicity, carcinogenicity, and mutagenic activity of paeoniflorin, paeoniflorin is not listed in EPA's Toxic Substances Control Act (TSCA) Inventory (NLM, 2000a).

No reports of occupational exposure to paeoniflorin during its production or processing were found in the available literature. No listing was found for paeoniflorin in the National Occupational Exposure Survey (NOES), which was conducted by the National Institute for Occupational Safety and Health (NIOSH) between 1981 and 1983.

No standards or guidelines have been set by NIOSH or OSHA for occupational exposure to or workplace allowable levels of paeoniflorin. Paeoniflorin was not on the American Conference of Governmental Industrial Hygienists (ACG1H) list of compounds for which recommendations for a threshold limit value (TLV) or biological exposure index (BEI) are made. No epidemiological studies or case reports investigating the association of exposure to paeoniflorin and cancer risk in humans were identified in the available literature.

The study of acute paeoniflorin toxicity in rodents (28) demonstrated the following results: Acute toxicity (LD50): LD50:Lethal dose, 50 percent kill. Intraperitoneal. Rodent-mouse. 3530 mg/kg. Toxic Effects: Behavioral-somnolence (general depressed activity). LD50: Lethal dose, 50 percent kill. Intravenous.Rodent-mouse. 9530 mg/kg. Toxic Effects: Behavioral-sleep. Behavioral-somnolence (general depressed activity). No data on chronic paeoniflorin toxicity in animals are available. No carcinogenicity studies of paeoniflorin were identified in the available literature.

In the reverse mutation assay, paeoniflorin was found to be nonmutagenic in the Bacillus subtilis rec assay and Salmonella microsome reversion assay (29).

Regarding human use, peony root extract containing paeoniflorin has been used for centuries as a traditional Chinese remedy (alone and in combination with licorice root extract) to treat muscle cramps, spasms and muscle tension. The efficacy of peony extract in relieving muscle cramps/muscle pain is supported by several clinical trials. These included patients with muscle pain associated with liver cirrhosis (30), diabetes mellitus (31), chronic renal insufficiency on hemodialysis (32), chronic alcohol abuse (33), cerebrovascular disease (34) and in cancer patients who suffered from muscle pain due to chemotherapy with paclitaxel and carboplatin (35).

Due to its anti-inflammatory activity, peony root extract was shown to benefit patients with rheumatoid arthritis and amplify the therapeutic effects of such known anti-rheumatic drugs as leflunomide and methotrexate. To observe the effect of total glucosides of peony (TGP) combined with methotrexate (MTX) on rheumatoid arthritis (RA) was the objective of the following study. In an open labeled clinical trial, 180 patients with rheumatoid arthritis were treated with methotrexate and peony root extract and compared to 80 patients treated with methotrexate and sulfasalazine. Administration of methotrexate in combination with the peony root extract resulted in a more rapid mode of improvement, fewer side effects and higher compliance rate as compared to the methotrexate plus sulfasalazine treated group. Coadministration of peony root extract in combination with leflunomide (40 patients) was superior to leflunomide alone (40 patients) in regards to rheumatoid arthritis clinical symptom and laboratory findings improvement.

Peony root extract is also utilized in oriental medicine as a hepatoprotective remedy to treat patients with chronic viral hepatitis. In one small, open clinical trial using red peony over a three-month period, 77 percent of patients with cirrhosis or chronic active hepatitis experienced improvement in liver histology based on repeat biopsy results (36). A case series also reported the efficacy of this approach (37).

In traditional Chinese medicine peony root extract is commonly used for various women's health problems, including dysmenorrheal (38, 39), polycystic ovary syndrome (40) luteal dysfunction and menopausal symptoms (41).

Several studies suggest that peony root extract may be beneficial in patients with atherosclerosis and/or hypertension due to its antiplatelet and anti-atherosclerotic properties (42, 43).

Contraindications for the use of peony extract or paeoniflorin are few. They include pregnancy, lactation, muscle weakness, hereditary myopathies and muscle dystrophies, myasthenia gravis, and opioid-induced constipation.

Adverse reactions associated with the use of peony extract or paeoniflorin are typically mild. They include allergic reactions, somnolence, muscle weakness, constipation, hypotension, and gastrointestinal discomfort and abdominal pain.

Potential drug interactions include possible interactions with muscle relaxants, L-dopa, Warfarin, neurontin (gabapentin), Lyrica, spasmolytics, antibiotics, calcium channel Mockers, ACE inhibitors, and beta blockers.

Paeoniflorin should not be used with the following herbal remedies: Veratum album, valerian root, rhubarb (Rheum officinale), Cuscuta japonica, or Fritillaria verticillate.

Regarding potential clinical applications of peony extract and paeoniflorin in rheumatology, paeoniflorin is a dietary supplement. Since 1994, dietary supplements have been regulated under the Dietary Supplement Health and Education Act (DSHEA). The DSHEA requires no proof of safety for dietary supplements on the market prior to Oct. 15, 1994. Labeling requirements for dietary supplements allow warnings and dosage recommendations as well as substantiated “structure or function” claims. All claims must prominently note that they have not been evaluated by the FDA, and they must bear the statement “This product is not intended to diagnose, treat, cure, or prevent any disease” (FDA. 1995).

Based on the known biological effects of paeoniflorin, this remedy has a potential for patients with muscle spasms, myofascial pain, fibromyalgia, and chronic low back pain. Furthermore, based on its neuromodulating activity, paeoniflorin can be viewed as a promising supplement for patients with cognitive dysfunction and “brain fog” associated with lupus, gluten intolerance and fibromyalgia. Anti-inflammatory activities of paeoniflorin in vitro and in vivo suggest that it may also be of value in patients with various inflammatory conditions, including hepatitis-associated arthropathies and myopathies, ankylosing spondylitis, psoriatic arthritis, arthropathies associated with ulcerative colitis and Crohn's disease, rheumatoid arthritis, and inflammatory osteoarthritis.

Bioperin, also known as piperine, is a black pepper extract that is a known inhibitor of glucuronidase in the gastrointestinal tract and in the liver. Bioperin is also a stimulator of thermogenesis. Bioperin has been shown to increase the bioavailability of a large number of drugs, including propranolol. The properties of bioperin are described in U.S. Pat. No. 5,744,161 to Majeed et al. and U.S. Pat. No. 5,972,382 to Majeed et al., both of which are incorporated herein in their entirety by this reference.

As indicated above, one aspect of the invention is a composition including both harpagoside and paeoniflorin. Harpagoside or crude herbal extracts containing harpagoside and paeoniflorin or crude herbal extracts containing paeoniflorin are known to possess anti-inflammatory activity and have been used to treat pain and inflammation. This invention provides the combination of harpagoside and paeoniflorin as a new herbal remedy with beneficial therapeutic activities to treat pain, inflammation, arthritis, muscle spasms and headache.

The specific benefits of this combination include: more rapid onset of action as compared to each of the individual ingredients, synergistic analgesic and anti-inflammatory activities, reduction of the frequency of gastrointestinal side effects, and synergistic muscle relaxing activity.

The invention also covers a range of harpagoside concentrations from about 1 mg to about 500 mg in combination with paeoniflorin range of concentrations from about 1 mg to about 1000 mg suitable for consumption on a daily basis. The combination can be used via oral, transcutaneous, parenteral, or intraarticular routes of administration.

Accordingly, in general, one embodiment of the present invention is a pharmaceutical composition comprising:
(1) a therapeutically effective quantity of harpagoside; and
(2) a therapeutically effective quantity of paeoniflorin, in a unit dose.

Typically, the pharmaceutical composition further comprises a pharmaceutically acceptable carrier, diluent, or excipient.

In general, the pharmaceutical composition typically comprises from about 1 mg to about 500 mg, of harpagoside and from about 1 mg to about 1000 mg of paeoniflorin as a unit dose.

The pharmaceutical composition can be formulated for oral, transcutaneous, parenteral, or intraarticular routes of administration.

Typically, the pharmaceutical composition is formulated for the treatment of a disease or condition selected from the group consisting of pain, inflammation, arthritic conditions and other chronic rheumatic diseases, muscle spasms, and headache.

Pharmaceutically acceptable carriers, diluents, or excipients are agents which are not biologically or otherwise undesirable, i.e., the agents can be administered to a subject along with the harpagoside and paeoniflorin, or other combinations of active ingredients as described herein without causing any undesirable biological effects or interacting in a deleterious manner with any of the components of the pharmaceutical composition in which it is contained. Pharmaceutically acceptable carriers enhance or stabilize the composition, or can facilitate preparation of the composition. Pharmaceutically acceptable carriers include solvents, dispersion media, coatings, antibacterial and antifungal agents, isotonic and absorption delaying agents, and the like that are physiologically compatible. The pharmaceutically acceptable carrier should be suitable for various routes of administration described herein.

Pharmaceutical compositions of the invention can be prepared in accordance with methods well known and routinely practiced in the art. See, e.g., Remington: The Science and Practice of Pharmacy, Mack Publishing Co., 20^{th} ed., 2000; and Sustained and Controlled Release Drug Delivery Systems, J. R. Robinson, ed., Marcel Dekker, Inc., New York, 1978. Pharmaceutical compositions are preferably manufactured under GMP conditions. Formulations for parenteral administration may, for example, contain excipients, sterile water, or saline, polyalkylene glycols such as polyethylene glycol, oils of vegetable origin, or hydrogenated naphthalenes. Biocompatible, biodegradable lactide polymers, lactide/glycolide copolymers, or polyoxyethylene-polyoxypropylene copolymers may be used to control the release of the compounds. Other potentially useful parenteral delivery systems for molecules of the invention include ethylene-vinyl acetate copolymer particles, osmotic pumps, implantable infusion systems, and liposomes.

Preparations formulated for oral administration may he in the form of tablets, dragees, capsules, or solutions. The pharmaceutical compositions contemplated by the present invention may be manufactured in a manner that is itself known, e.g., by means of conventional mixing, dissolving, granulating, dragee-making, levitating, emulsifying, encapsulating, entrapping or lyophilizing processes.

Pharmaceutical formulations for parenteral administration include aqueous solutions of the active ingredients in water-soluble form. Additionally, suspensions of the active ingredients can be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or modulators which increase the solubility of the active ingredients to allow for the preparation of highly concentrated solutions. Pharmaceutical preparations for oral use can be obtained by combining the active modulators with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating modulators may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different doses of active ingredients.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added.

Other ingredients such as stabilizers, for example, antioxidants such as sodium citrate, ascorbyl palmitate, propyl gallate, reducing agents, ascorbic acid, vitamin E, sodium bisulfite, butylated hydroxytoluene, BHA, acetylcysteine, monothioglycerol, phenyl-α-naphthylamine, or lecithin can be used. Also, chelators such as EDTA can be used. Other ingredients that are conventional in the area of pharmaceutical compositions and formulations, such as lubricants in tablets or pills, coloring agents, or flavoring agents, can be used. Also, conventional pharmaceutical excipients or carriers can be used. The pharmaceutical excipients can include, but are not necessarily limited to, calcium carbonate, calcium phosphate, various sugars or types of starch, cellulose derivatives, gelatin, vegetable oils, polyethylene glycols and physiologically compatible solvents. Other pharmaceutical excipients are well known in the art. Exemplary pharmaceutically acceptable carriers include, but are not limited to, any and/or all of solvents, including aqueous and non-aqueous solvents, dispersion media, coatings, antibacterial and/or antifungal agents, isotonic and/or absorption delaying agents, and/or the like. The use of such media and/or agents for pharmaceutically active substances is well known in the art. Except insofar as any conventional medium, carrier, or agent is incompatible with the active ingredient or ingredients, its use in a composition according to the present invention is contemplated. Supplementary active ingredients can also be incorporated into the compositions, particularly as described above. For administration of any of the compounds used in the present invention, preparations should meet sterility, pyrogenicity, general safety, and purity standards as required by the FDA Office of Biologics Standards or by other regulatory organizations regulating drugs.

Sustained-release formulations or controlled-release formulations are well-known in the art. For example, the sustained-release or controlled-release formulation can be (1) an oral matrix sustained-release or controlled-release formulation; (2) an oral multilayered sustained-release or controlled-release tablet formulation; (3) an oral multiparticulate sustained-release or controlled-release formulation; (4) an oral osmotic sustained-release or controlled-release formulation; (5) an oral chewable sustained-release or controlled-release formulation; or (6) a dermal sustained-release or controlled-release patch formulation.

The pharmacokinetic principles of controlled drug delivery are described, for example. in B. M. Silber et al., “Pharmacokinetic/Pharmacodynamic Basis of Controlled Drug Delivery” in Controlled Drug Delivery: Fundamentals and Applications (J. R. Robinson & V. H. L. Lee, eds, 2d ed., Marcel Dekker, New York, 1987), ch. 5, pp. 213-251, incorporated herein by this reference.

One of ordinary skill in the art can readily prepare formulations for controlled release or sustained release comprising the active ingredients by modifying the formulations described above, such as according to principles disclosed in V. N. K. Li et al, “Influence of Drug Properties and Routes of Drug Administration on the Design of Sustained and Controlled Release Systems” in Controlled Drug Delivery: Fundamentals and Applications (J. R. Robinson & V. H. L. Lee, eds, 2d ed., Marcel Dekker, New York, 1987), ch. 1, pp. 3-94, incorporated herein by this reference. This process of preparation typically takes into account physicochemical properties of the active ingredients, such as aqueous solubility, partition coefficient, molecular size, stability of the active ingredients, and binding of the active ingredients to proteins and other biological macromolecules. This process of preparation also takes into account biological factors, such as absorption, distribution, metabolism, duration of action, the possible existence of side effects, and margin of safety, for the active ingredients. Accordingly, one of ordinary skill in the art could modify the formulations in order to incorporate the active ingredients into a formulation having the desirable properties described above for a particular application.

Another embodiment of the present invention, as described above, includes bioperin in a therapeutically effective quantity in the unit dose. The bioperin is included in a therapeutically effective quantity to improve bioavailability of the harpagoside and paeoniflorin. A therapeutically effective quantity of bioperin is typically from about 0.1 mg to about 20 mg per unit dose.

Therefore, in general, this embodiment of the present invention is a pharmaceutical composition comprising:
(1) a therapeutically effective quantity of harpagoside;
(2) a therapeutically effective quantity of paeoniflorin; and
(3) a therapeutically effective quantity of bioperin, in a unit dose.

In a preferred embodiment the pharmaceutical composition comprises from about 10 mg to about 250 mg of harpagoside, more preferably from about 50 mg to about 175 mg of harpagoside and most preferably about 125 mg of harpagoside, in combination with from about 10 mg to about 500 mg of paeoniflorin, more preferably from about 50 mg to about 200 mg of paeoniflorin and most preferably about 100 mg of paeoniflorin, in further combination with from about 0.5 mg to about 10 mg of bioperin, more preferably from about 1 mg to about 5 mg of bioperin and most preferably about 2.5 mg of bioperin as a unit dose.

Typically, as described above, this embodiment of the pharmaceutical composition also further comprises a pharmaceutically acceptable carrier, diluent, or excipient.

Yet another embodiment of the present invention is a pharmaceutical composition incorporating harpagoside and bioperin.

In general, this pharmaceutical composition comprises:
(1) a therapeutically effective quantity of harpagoside; and
(2) a therapeutically effective quantity of bioperin, in a unit dose.

Typically, as described above, this embodiment of the pharmaceutical composition also further comprises a pharmaceutically acceptable carrier, diluent, or excipient.

In this embodiment of the composition, the pharmaceutical composition typically comprises from about 1 mg to about 500 mg of harpagoside per unit dose and from about 0.1 mg to about 20 mg of bioperin per unit dose.

Yet another embodiment of the present invention is a pharmaceutical composition incorporating paeoniflorin and bioperin.

In general, this pharmaceutical composition comprises:
(1) a therapeutically effective quantity of paeoniflorin; and
(2) a therapeutically effective quantity of bioperin, in a unit dose.

Typically, as described above, this embodiment of the pharmaceutical composition also further comprises a pharmaceutically acceptable carrier, diluent, or excipient.

In this embodiment of the composition, the pharmaceutical composition typically comprises from about 1 mg to about 1000 mg of paeoniflorin per unit dose and from about 0.1 mg to about 20 mg of bioperin per unit dose.

Another aspect of the present invention is a purified preparation of harpagoside that comprises at least 45% harpagoside. Typically, the proportion of harpagoside is determined by high performance liquid chromatography (HPLC).

Yet another aspect of the present invention is a method for treating a patient who has a condition selected from the group consisting of pain, inflammation, arthritic conditions and other chronic rheumatic diseases, muscle spasms, and headache.

In general, this method comprises the step of administering a therapeutically effective dose of a pharmaceutical composition according to the present invention, as described above, to the patient to treat the patient.

### ADVANTAGES OF THE INVENTION

In order to determine the efficacy of the present invention, a trial was conducted. Nineteen patients from three chiropractic doctors and medical doctor were selected to participate in the study. Among the patients there were 13 females and 6 males varying in age from 41 to 83. All the patients were suffering from some for of arthritic pain. The Mosby Pain Scale© shown below was used to rate the level of pain each patient was experiencing throughout the trial.

TABLE 2
Mosby Pain Scale

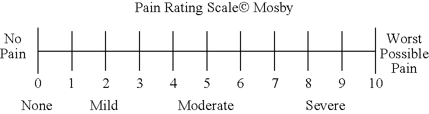

| | | | | | |
|---|---|---|---|---|---|
| [symbol] | [symbol] | [symbol] | [symbol] | [symbol] | [symbol] |
| 0 | 2 | 4 | 6 | 8 | 10 |
| NO HURT | HURTS | HURTS | HURTS | HURTS | HURTS |
| | LITTLE BIT | LITTLE MORE | EVEN MORE | WHOLE LOT | WORST |

Patients received two doses of two tablets daily for four weeks. A single dose consists of two tablets. Each tablet contains 125 mg Devil's Claw Root Extract, 100 mg Paeoniflorin, and 2.5 mg Bioperene. Table 3 shows pain ratings patients each patient assigned for a given week. The pre-trial average for the beginning pain scale rating was 6.4. The post trial average after four weeks was 3.2. Of the ten patients that provided weekly pain ratings the average amount of time to reach the lowest pain level was 2.3 weeks.

TABLE 3
Results of patients after receiving pharmaceutical composition for treatment of pain.

| Doctor | Age | Gender | Beginning | Week 1 | Week 2 | Week 3 | Week 4 | Change | Weeks to low |
|---|---|---|---|---|---|---|---|---|---|
| Johnson | 59 | F | 5 | 3 | 3 | 2 | 2 | −3 | 3 |
| Johnson | 45 | F | 4 | 2 | 2 | 2 | 2 | −2 | 1 |
| Johnson | 60 | F | 4 | 2 | 2 | 2 | 2 | −2 | 1 |
| Johnson | 58 | F | 3 | 2 | 1 | 1 | 1 | −2 | 2 |
| Ninberg | 54 | M | 5 | 6 | 4 | 4 | 4 | −1 | 2 |
| Ninberg | 49 | M | 7 | | | | 4 | −3 | |
| Ninberg | 41 | M | 6 | 6 | 4 | 2 | 2 | −4 | 3 |
| Shikhman | 83 | F | 8 | | | | 4.5 | −3.5 | |
| Shikhman | 77 | M | 6.5 | | | | 3.5 | −3 | |
| Shikhman | 55 | M | 6.5 | | | | 2.5 | −4 | |
| Shikhman | 62 | F | 7.5 | | | | 3.5 | −4 | |
| Shikhman | 53 | F | 6.5 | | | | 5.5 | −1 | |
| Shikhman | 71 | F | 6.5 | | | | 5.5 | −1 | |
| Shikhman | 45 | M | 8 | | | | 2.5 | −5.5 | |
| Shikhman | 43 | F | 7.5 | | | | 3.5 | −4 | |
| Scott | 80 | F | 6.5 | 6.5 | 4 | 1 | 1 | −5.5 | 3 |
| Scott | 47 | F | 8 | 8 | 7 | 4.5 | 4.5 | −3.5 | 3 |
| Scott | 64 | F | 8 | 6 | 7 | 5 | 5 | −3 | 3 |
| Scott | 70 | F | 7.5 | 1 | 2 | 2 | 2 | −5.5 | 2 |
| Average | 58.7 | | 6.4 | | | | 3.2 | −3.2 | 2.3 |

As used herein, the terms “treating” or similar terminology do not imply a cure for any of the diseases or conditions described above, i.e., pain, inflammation, arthritic conditions and other chronic rheumatic diseases, muscle spasms, and headache, or any other disease or condition; rather, this terminology is used to refer to any clinically detectable improvement in the disease or condition being treated or alleviated, including, but not limited to, reduction in pain, reduction in inflammation, improvement in joint mobility, improvement in subjective well-being experienced by the patient, or any other clinically detectable improvement. As used herein, the term “therapeutically effective amount” refers to the amount of a therapy (e.g., a prophylactic or therapeutic agent) which is sufficient to treat any of the diseases or conditions described above.

The pharmaceutical composition in the methods of the invention is typically administered to a subject in an amount that is sufficient to achieve the desired therapeutic effect in a subject in need thereof. The selected dosage level for pharmaceutical compositions according to the present invention depends upon a variety of pharmacokinetic factors including the concentration of the active agents in the pharmaceutical composition, the route of administration, the frequency of administration, the rate of excretion of the active agents, the severity of the condition, other health considerations affecting the subject, and the status of liver and kidney function of the subject. It also depends on the duration of the treatment, other drugs; compounds and/or materials used in combination with pharmaceutical compositions according to the present invention, as well as the age, weight, condition, general health and prior medical history of the subject being treated, and like factors. Methods for determining optimal dosages are described in the art, e.g., Remington: The Science and Practice of Pharmacy, Mack Publishing Co., 20^{th} ed., 2000.

Pharmaceutical compositions and methods according to the present invention provide improved treatment modalities for a number of diseases and conditions, such as pain, inflammation, arthritic conditions and other chronic rheumatic diseases, muscle spasms, and headache. They are well tolerated, have few side effects, and can he used with other medications for these diseases and conditions as well as lifestyle changes such as improved diet, exercise programs, and programs of relaxation and stress reduction.

Treatment methods according to the present invention possess industrial applicability for the preparation of a medicament for the treatment of diseases and conditions such as pain, inflammation, arthritic conditions and other chronic rheumatic diseases, muscle spasms, and headache.

With respect to ranges of values, the invention encompasses each intervening value between the upper and lower limits of the range to at least a tenth of the lower limit's unit, unless the context clearly indicates otherwise. Moreover, the invention encompasses any other stated intervening values and ranges including either or both of the upper and lower limits of the range, unless specifically excluded from the stated range.

Unless defined otherwise, the meanings of all technical and scientific terms used herein are those commonly understood by one of ordinary skill in the art to which this invention belongs. One of ordinary skill in the art will also appreciate that any methods and materials similar or equivalent to those described herein can also be used to practice or test this invention.

The publications and patents discussed herein are provided solely for their disclosure prior to the filing date of the present application. Nothing herein is to be construed as an admission that the present invention is not entitled to antedate such publication by virtue of prior invention. Further the dates of publication provided may be different from the actual publication dates which may need to be independently confirmed.

All the publications cited are incorporated herein by reference in their entireties, including all published patents, patent applications, literature references, as well as those publications that have been incorporated in those published documents. However, to the extent that any publication incorporated herein by reference refers to information to be published, applicants do not admit that any such information published after the filing date of this application to be prior art.

As used in this specification and in the appended claims, the singular forms include the plural forms. For example the terms “a,” “an,” and “the” include plural references unless the content clearly dictates otherwise. Additionally, the term “at least” preceding a series of elements is to be understood as referring to every element in the series. The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein. In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. Those skilled in the art will recognize, or will be able to ascertain using no more than routine experimentation, many equivalents to the specific embodiments of the invention described. Such equivalents are intended to be encompassed by the following claims.

### INCORPORATION BY REFERENCE

Throughout this application, various publications, patents, and/or patent applications are referenced in order to more fully describe the state of the art to which this invention pertains. The disclosures of these publications, patents, and/or patent applications are herein incorporated by reference in their entireties, and for the subject matter for which they are specifically referenced in the same or a prior sentence, to the same extent as if each independent publication, patent, and/or patent application was specifically and individually indicated to be incorporated by reference.

### OTHER EMBODIMENTS

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims

### REFERENCES

1. Tsuboi H, Hossain K, Akhand A A, Takeda K, Du J, Rifa'i M, Dai Y, Hayakawa A, Suzuki H, Nakashima I. Paeoniflorin induces apoptosis of lymphocytes through a redox-linked mechanism. J Cell Biochem. 2004 Sep. 1; 93(1): 162-72.

2. Wu H, Wei W, Song L, Zhang L, Chen Y, Hu X. Paeoniflorin induced immune tolerance of mesenteric lymph node lymphocytes via enhancing beta 2-adrenergic receptor desensitization in rats with adjuvant arthritis. Int Immunopharmacol. 2007 May; 7(5): 662-73.

3. Chu D, Luo Q. Li C, Gao Y, Yu L, Wei W, Wu Q, Shen J. Paeoniflorin inhibits TGF-betal-mediated collagen production by Schistosoma japonicum soluble egg antigen in vitro. Parasitology. 2007 October; 134 (Pt 11):1611-21.

4. Li C R, Zhou Z, Zhu D, Sun Y N, Dai J M, Wang S Q. Protective effect of paeoniflorin on irradiation-induced cell damage involved in modulation of reactive oxygen species and the mitogen-activated protein kinases. Int J Biochem Cell Biol. 2007; 39(2): 426-38.

5. Tsai TY, Wu S N, Liu Y C, Wu A Z, Tsai Y C. Inhibitory action of L-type Ca2+ current by paeoniflorin, a major constituent of peony root, in NG108-15 neuronal cells. Eur J Pharmacol. 2005 Oct. 31; 523(1-3): 16-24

6. Tang L M, Liu I M, Cheng J T. Stimulatory effect of paeoniflorin on adenosine release to increase the glucose uptake into white adipocytes of Wistar rat. Planta Med. 2003 April; 69(4): 332-6.

7. Zheng Y Q, Wei W, Zhu L, Liu J X. Effects and mechanisms of Paeoniflorin, a bioactive glucoside from paeony root, on adjuvant arthritis in rats. Inflamm Res. 2007 May; 56(5): 182-8.

8. Liu D F, Wei W. Song L H. Protective effect of paeoniflorin on immunological liver injury induced by bacillus Calmette-Guerin plus lipopolysaccharide: modulation of tumour necrosis factor-alpha and interleukin-6 MRNA. Clin Exp Pharmacol Physiol. 2006 April; 33(4): 332-9.

9. Dezaki K, Kimura I, Miyahara K, Kimura M. Complementary effects of paeoniflorin and glycyrrhizin on intracellular Ca2+ mobilization in the nerve-stimulated skeletal muscle of mice. Jpn J Pharmacol. 1995 November; 69(3): 281-4.

10. Tamaya T, Sato S, Okada H H. Possible mechanism of steroid action of the plant herb extracts glycyrrhizin, glycyrrhetinic acid, and paeoniflorin: inhibition by plant herb extracts of steroid protein binding in the rabbit. Am J Obstet Gynecol. 1986 November; 155(5): 1134-9.

11. Goto H, Shimada Y, Akechi Y, Kohta K, Hattori M, Terasawa K. Endothelium-dependent vasodilator effect of extract prepared from the roots of Paeonia lactiflora on isolated rat aorta. Planta Med. 1996 October; 62(5): 436-9.

12. Ye J, Duan H. Yang X. Yan W, Zheng X. Anti-thrombosis effect of paeoniflorin: evaluated in a photochemical reaction thrombosis model in vivo. Planta Med. 2001 November; 67(8): 766-7.

13. Lee S, Lim J M, Jin M H, Park H K, Lee E J, Kang S, Kim Y S, Cho W G. Partially purified paeoniflorin exerts protective effects on UV-induced DNA damage and reduces facial wrinkles in human skin. J Cosmet Sci. 2006 January-February; 57(1): 57-64.

14. Takagi K, Harada M. [Pharmacological studies on herb paeony root. II. Anti-inflammatory effect, inhibitory effect on gastric juice secretion, preventive effect on stress ulcer, antidiuretic effect of paeoniflorin and combined effects with licorice component Fm 100] Yakugaku Zasshi. 1969 July; 89(7): 887-92.

15. Hsu F L, Lai C W, Cheng J T. Antihyperglycemic effects of paeoniflorin and 8-debenzoylpaeoniflorin, glucosides from the root of Paeonia lactiflora. Planta Med. 1997 August; 63(4): 323-5.

16. Yang H O, Ko W K, Kim J Y, Ro H S. Paeoniflorin: an antihyperlipidemic agent from Paeonia lactiflora. Fitoterapia. 2004 January; 75(1):45-9.

17. Xiao L, Wang Y Z, Liu J, Luo X T, Ye Y, Zhu X Z. Effects of paeoniflorin on the cerebral infarction, behavioral and cognitive impairments at the chronic stage of transient middle cerebral artery occlusion in rats. Life Sci. 2005 Dec. 12; 78(4): 413-20.

18. Yu H Y, Liu M G, Liu D N, Shang G W, Wang Y, Qi C, Zhang K P, Song Z J, Chen J. Antinociceptive effects of systemic paeoniflorin on bee venom-induced various ‘phenotypes’ of nociception and hypersensitivity. Pharmacol Biochem Behav. 2007 December; 88(2): 131-40.

19. Liu D Z, Zhu J. Jin D Z, Zhang L M, Ji X Q, Ye Y, Tang C P, Zhu X Z. Behavioral recovery following sub-chronic paeoniflorin administration in the striatal 6-OHDA lesion rodent model of Parkinson's disease. J Ethnopharmacol. 2007 Jun. 13; 112(2): 327-32.

20. Abdel-Hafez A A, Meselhy M R, Nakamura N, Hattori M, Watanabe H, Murakami Y, El-Gendy M A, Mahfouz N M, Mohamed T A. Anticonvulsant activity of paeonimetabolin-I adducts obtained by incubation of paeoniflorin and thiol compounds with Lactobacillus brevis. Biol Pharm Bull. 1999 May; 22(5): 491-7.

21. Watanabe H. Candidates for cognitive enhancer extracted from medicinal plants: paeoniflorin and tetramethylpyrazine. Behav Brain Res. 1997 February; 83(1-2): 135-41.

22. Liu J, Jin D Z, Xiao L, Zhu X Z. Paeoniflorin attenuates chronic cerebral hypoperfusion-induced learning dysfunction and brain damage in rats. Brain Res. 2006 May 17; 1089(1): 162-70.

23. Ohta H, Matsumoto K, Shimizu M, Watanabe H. Paeoniflorin attenuates learning impairment of aged rats in operant brightness discrimination task. Pharmacol Biochem Behav. 1994 September; 49(1): 213-7.

24. Takeda S, Isono T, Wakui Y, Matsuzaki Y, Sasaki H, Amagaya S, Maruno M. Absorption and excretion of paeoniflorin in rats. J Pharm Pharmacol. 1995 December; 47(12A): 1036-40.

25. Ju-Xiu H E, Teruaki AKAO, and Tadato TANI. Restorative Effect of Repetitive Administration of Shaoyao-Gancao-tang on Bioavailability of Paeoniflorin Reduced by Antibacterial Synthetic Drugs Treatment in Rats Biol. Pharm. Bull. 26(11) 1585-1590 (2003).

26. Takeda S, Isono T, Wakui Y, Mizuhara Y, Amagaya S, Maruno M, Hattori M. In-vivo assessment of extrahepatic metabolism of paeoniflorin in rats: relevance to intestinal floral metabolism. J Pharm Pharmacol. 1997 January; 49(1):35-9.

27. Chen L C, Chou M H, Lin M F, Yang L L. Pharmacokinetics of paeoniflorin after oral administration of Shao-yao Gan-chao Tang in mice. Jpn J Pharmacol. 2002 March; 88(3):250-5.

28. YKKZAJ Yakugaku Zasshi. Journal of Pharmacy. (Nippon Yakugakkai, 2-12-15 Shibuya, Shibuya-ku, Tokyo 150, JP) No. 1-1881-Volume(issue)/page/year: 89, 899, 1969.

29. Morimoto I et al. Mutagenicity screening of crude drugs with Bacillus subtilis recassay and Salmonella microsome reversion assay. Mutation research, 1982, 97:81-102.

30. Kumada T, et al. Effect of shakuyaku-kanzo-to (Tsumura TJ-68) on muscle cramps accompanying cirrhosis in a placebocontrolled double-blind parallel study. J. Clin Ther Med 1999; 15: 499-523.

31. Yosida M, et al. Effects of shakuyaku-kanzo-to on muscle cramp in diabetics. Neurol Ther 1995; 12:529-534.

32. Yamashita J I. Effect of Tsumura skakuyaku-kanzo-to on pain at muscle twitch during and after dialysis in the patients undergoing dialysis. Pain & Kampo Medicine 1992; 2:18-20.

33. Maruyama K, et al. Effectiveness of shakuyaku-kanzo-to on convulsion and pain associated with alcohol dependence. Kampo Igaku 1996; 20: 81-84.

34. Sakamoto T, Hosino M. Effect of shakuyaku-kanzo-to extract granules on convulsion of gastrocnemius muscle in patients with cerebrovascular disorder. Jpn J Oriental Med 1995; 45: 563-568.

35. Yamamoto K, Hoshiai H, Noda K. Effects of shakuyaku-kanzo-to on muscle pain from combination chemotherapy with paclitaxel and carboplatin, Gynecol Oncol 2001; 81: 333-334.

36. Yang D G. Comparison of pre- and post-treatment hepatohistology with heavy dosage of Paeonia rubra on chronic active hepatitis caused liver fibrosis. Chung Kuo Chung Hsi I Chieh Ho Tsa Chih 1994; 14: 207-209, 195.

37. Wang C B, Chang A M. Plasma thromboxane B2 changes in severe icteric hepatitis treated by traditional Chinese medicine—dispelling the pathogenic heat from blood, promoting blood circulation and administrating large doses of radix paeoniae—a report of 6 cases. Chung Hsi I Chieh Ho Tsa Chih 1985; 5: 326-328, 322.

38. Kotani N, Oyama T, Hashimoto H, et al. Analgesic effect of a herbal medicine for treatment of primary dysmenorrhea—a double-blind study. Am J Chin Med 1997; 25: 205-212.

39. Sakamoto S, Mitamura T, lwasawa M, et al. Conservative management for perimenopausal women with uterine leiomyomas using Chinese herbal medicines and synthetic analogs of gonadotropin-releasing hormone. In Vivo 1998; 12: 333-337.

40. Takahashi K, Kitao M. Effect of TJ-68 (shakuyaku-kanzo-to) on polycystic ovarian disease. Int J Fert Menopausal Studies 1994; 39: 69-76.

41. Hagino N. An overview of Kampo medicine: Toki-Shakuyaku-San (TJ-p23). Phytother Res 1993; 7: 391-394.

42. Liu J. Effect of Paeonia obovata 801 on metabolism of thromboxane B2 and arachidonic acid and on platelet aggregation in patients with coronary heart disease and cerebral thrombosis. Chung Hua I Hsueh Tsa Chih 1983; 63: 477-481.

43. Guo T L, Zhou X W. Clinical observations on the treatment of the gestational hypertension syndrome with angelica and paeonia powder. Chung Hsi I Chieh Ho Tsa Chih 1986; 6: 714-716, 707.

44. Yamada K, Kanba S, Murata T, et al. Effectiveness of skakuyaku-kanzo-to in neuroleptic-induced hyperprolactinemia: a preliminary report. Psychiatr Clin Neurosci 1996; 50: 341-342.

45. Yamada K, Kanba S, Yagi G, Asai M. Effectiveness of herbal medicine (skakuyaku-kanzo-to) for neuroleptic-induced hyperolactinemia. J Clin Psychopharmacol 1997; 17: 234-235.

46. Yamada K, Kanba S, Yagi G, Asai M. Herbal medicine (skakuyaku-kanzo-to) in the treatment of risperidone-induced amenorrhea. J Clin Psychopharmacol 1999; 19: 380-381.

47. Tanaka T. Effects of herbal medicines on menopausal symptoms induced by gonadotropin-releasing hormone agonist therapy. Clin Exp Obstet Gynecol 2001; 28: 20-23.

What is claimed:

1. A pharmaceutical composition comprising:
(a) a therapeutically effective quantity of harpagoside; and
(b) a therapeutically effective quantity of paeoniflorin, in a unit dose.

2. The pharmaceutical composition of claim 1 further comprising a pharmaceutically acceptable carrier, diluent, or excipient.

3. The pharmaceutical composition of claim 1 wherein the composition comprises from about 1 mg to about 500 mg of harpagoside as a unit dose.

4. The pharmaceutical composition of claim 1 wherein the composition comprises from about 1 mg to about 1000 mg of paeoniflorin as a unit dose.

5. The pharmaceutical composition of claim 3 wherein the composition comprises from about 1 mg to about 1000 mg of paeoniflorin as a unit dose.

6. The pharmaceutical composition of claim 1 wherein the pharmaceutical composition is formulated for the treatment of a disease or condition selected from the group consisting of pain, inflammation, arthritic conditions and other chronic rheumatic diseases, muscle spasms, and headache.

7. A pharmaceutical composition comprising:
(a) a therapeutically effective quantity of harpagoside;
(b) a therapeutically effective quantity of paeoniflorin; and
(c) a therapeutically effective quantity of bioperin, in a unit dose.

8. The pharmaceutical composition of claim 7 further comprising a pharmaceutically acceptable carrier, diluent, or excipient.

9. The pharmaceutical composition of claim 7 wherein the composition comprises from about 1 mg to about 500 mg of harpagoside as a unit dose.

10. The pharmaceutical composition of claim 7 wherein the composition comprises from about 1 mg to about 1000 mg of paeoniflorin as a unit dose.

11. The pharmaceutical composition of claim 7 wherein the composition comprises from about 0.1 mg to about 20 mg of bioperin as a unit dose.

12. The pharmaceutical composition of claim 7 wherein the composition comprises from about 1 mg to about 500 mg of harpagoside, from about 1 mg to about 1000 mg of paeoniflorin, and from about 0.1 mg to about 20 mg of bioperin as a unit dose.

13. The pharmaceutical composition of claim 7 wherein the pharmaceutical composition is formulated for the treatment of a disease or condition selected from the group consisting of pain, inflammation, arthritic conditions and other chronic rheumatic diseases, muscle spasms, and headache.

14. A pharmaceutical composition comprising:
(a) a therapeutically effective quantity of harpagoside; and
(b) a therapeutically effective quantity of bioperin, in a unit dose.

15. The pharmaceutical composition of claim 14 further comprising a pharmaceutically acceptable carrier, diluent, or excipient.

16. The pharmaceutical composition of claim 14 wherein the composition comprises from about 1 mg to about 500 mg of harpagoside as a unit dose.

17. The pharmaceutical composition of claim 14 wherein the composition comprises from about 0.1 mg to about 20 mg of bioperin as a unit dose.

18. The pharmaceutical composition of claim 16 wherein the composition comprises from about 0.1 mg to about 20 mg of bioperin as a unit dose.

19. The pharmaceutical composition of claim 14 wherein the pharmaceutical composition is formulated for the treatment of a disease or condition selected from the group consisting of pain, inflammation, arthritic conditions and other chronic rheumatic diseases, muscle spasms, and headache.

20. A pharmaceutical composition comprising:
(a) a therapeutically effective quantity of paeoniflorin; and
(b) a therapeutically effective quantity of bioperin, in a unit dose.

21. The pharmaceutical composition of claim 20 further comprising a pharmaceutically acceptable carrier, diluent, or excipient.

22. The pharmaceutical composition of claim 20 wherein the composition comprises from about 1 mg to about 1000 mg of paeoniflorin as a unit dose.

23. The pharmaceutical composition of claim 20 wherein the composition comprises from about 0.1 mg to about 20 mg of bioperin as a unit dose.

24. The pharmaceutical composition of claim 22 wherein the composition comprises from about 0.1 mg to about 20 mg of bioperin as a unit dose.

25. The pharmaceutical composition of claim 20 wherein the pharmaceutical composition is formulated for the treatment of a disease or condition selected from the group consisting of pain, inflammation, arthritic conditions and other chronic rheumatic diseases, muscle spasms, and headache.

26. A method treating a patient who has a condition selected from the group consisting of pain, inflammation, arthritic conditions and other chronic rheumatic diseases, muscle spasms, and headache comprising the step of administering a therapeutically effective dose of the pharmaceutical composition of claim 1 to the patient to treat the patient.

27. A method treating a patient who has a condition selected from the group consisting of pain, inflammation, arthritic conditions and other chronic rheumatic diseases, muscle spasms, and headache comprising the step of administering a therapeutically effective dose of the pharmaceutical composition of claim 7 to the patient to treat the patient.

28. A method treating a patient Who has a condition selected from the group consisting of pain, inflammation, arthritic conditions and other chronic rheumatic diseases, muscle spasms, and headache comprising the step of administering a therapeutically effective dose of the pharmaceutical composition of claim 14 to the patient to treat the patient.

29. A method treating a patient who has a condition selected from the group consisting of pain, inflammation, arthritic conditions and other chronic rheumatic diseases, muscle spasms, and headache comprising the step of administering a therapeutically effective dose of the pharmaceutical composition of claim 20 to the patient to treat the patient.

30. A purified preparation of harpagoside that comprises at least 45% harpagoside.
